(19) United States
(12) Patent Application Publication
(10) Pub. No.: US 2007/0232995 A1
(43) Pub. Date: Oct. 4, 2007

(54) HOLLOW NEEDLE ASSEMBLY

(75) Inventor: James Samsoondar, Cambridge (CA)

Correspondence Address: BERESKIN AND PARR, 40 KING STREET WEST, BOX 401, TORONTO, ON, M5H 3Y2, CA

(73) Assignee: Chromedx Inc., Cambridge (CA)

(21) Appl. No.: 11/738,889

(22) Filed: Apr. 23, 2007

Related U.S. Application Data
(63) Continuation-in-part of application No. 11/466,588, filed on Aug. 23, 2006.

(30) Foreign Application Priority Data
Aug. 26, 2005 (CA) 2,517,299

(51) Int. Cl. A61M 31/00; A61M 5/32

(52) U.S. Cl. 604/93.01; 604/198

(57) ABSTRACT

Some embodiments of the invention provide a needle comprising a shaft with a sharp open end and a hub with a blunt open end, housed in a barrel with an open anterior end and an open posterior end. The hub of the needle can move forward inside the barrel, for extending the sharp open end of the shaft of the needle for insertion into a vessel, e.g. a blood vessel, a catheter, or a capped tube. After use, the hub can move backwards inside the barrel for retracting the shaft of the needle into the barrel. The blunt open end of the needle can be fluidly connected to the inlet opening of a measurement apparatus, so that the blood can flow directly from a vessel, into the measurement apparatus, without the use of a syringe. The hollow needle assembly can also be used with a traditional syringe, as an alternative to traditional needles, in order to minimize the risk of needle-stick injury.

### RELATED APPLICATIONS

This application is a continuation-in-part of U.S. patent application Ser. No. 11/466,588, filed Aug. 23, 2006, the entire contents of which are hereby incorporated by reference.

### FIELD OF THE INVENTION

The invention relates to a hollow needle assembly for transferring fluid from one site to another. In particular, the invention relates to the needle, and a barrel that facilitates extension and retraction of the sharp open end of the needle, in order to minimize the risk of needle-stick injury.

### BACKGROUND OF THE INVENTION

There are many medical diagnostic tests that require a blood sample. In general, conventional methods of collecting and analyzing blood leads to inevitable delays, unnecessary handling of the blood and the introduction of contaminants, which are all known sources of analysis error. More specifically, as per convention, a blood sample is typically withdrawn using one instrument/vessel and then transferred into another vessel for analysis. For example, a syringe is used to obtain a relatively large blood sample that is later injected into measuring instruments or disposable cartridges of measuring instruments. Syringe extraction of blood is beneficial in circumstances where several milliliters of blood are needed, and also in circumstances that require protection of the blood from atmospheric contamination. Alternatively, much smaller blood samples (e.g. in the range of micro-liters) can be obtained using a pinprick and then a capillary tube that is inserted into a drop of blood that oozes onto the skin surface. Blood from the drop flows into the capillary tube as a result of capillary action. Irrespective of the amount, collected blood is transferred into another vessel to be analyzed. The eventual transfer of blood between vessels delays the actual analysis of the blood sample and also exposes the blood sample to contaminants.

One example of a blood analysis technique that is affected by the aforementioned sources of error is co-oximetry. Co-oximetry is a spectroscopic technique that can be used to measure the different Hemoglobin (Hb) species present in a blood sample. The results of co-oximetry can be further evaluated to provide Hb Oxygen Saturation (Hb O₂ saturation) measurements. If the blood sample is exposed to air the Hb sO₂ saturation measurements are falsely elevated, as oxygen from the air is absorbed into the blood sample.

Another example of a blood analysis technique that is affected by the aforementioned sources of error is blood gases. Traditionally, blood gas measurement includes the partial pressure of oxygen, the partial pressure of carbon dioxide, and pH. From these measurements, other parameters can be calculated, for example, Hb O₂ saturation. Blood gas and electrolyte measurements usually employ biosensors. Bench-top analyzers are available, which (1) measure blood gases, (2) perform co-oximetry, or (3) measure blood gases and perform co-oximetry in combination. Some combinations of diagnostic measurement instruments also include electrolytes, making such instrument assemblies even larger. Because these instruments are large and expensive, they are usually located in central laboratories. Biosensor technology is also limited by the blood parameters it can measure. For example, biosensors are not currently available for measuring the Hb species measured by the available co-oximeters. Preferably, blood gases and co-oximetry are measured in arterial blood collected in a syringe, since arterial blood provides an indication of how well venous blood is oxygenated in the lungs. There are many benefits in providing these blood tests near or at the point of care of patients, but these are usually limited by the size and cost of the diagnostic measurement instruments.

In monitoring a patient's acid-base status, as a non-limiting example, an arterial blood sample is preferred. Arterial blood must be collected by a doctor or a specially-trained technician, using a syringe, because of a number of inherent difficulties associated with the complicated collection procedure. Notably, the collection of arterial blood is far more painful, difficult and dangerous for a patient, than the collection of venous blood. This is particularly true for infants. If a small sample of arterial blood (for example a fraction of a milliliter) can be used, a larger gauge needle (smaller outside diameter) could be used. The smaller the needle, the lower the level of trauma to the patient.

Needle-stick injury is common and the consequences can be fatal if a user sticks himself with a needle contaminated with blood from a patient who is infected with a deadly virus.

Even when it is convenient to use a syringe, for example when the patient has a catheter connected to an artery, the safety aspect of handling needles must be considered. Users are at risk of sticking their fingers with the needle, during removal of or recapping the needle. Recapping needles is an unsafe practice that is discouraged, and there is a need for improving the methods available for protecting users from needle-stick injuries.

### SUMMARY OF THE INVENTION

According to an aspect of an embodiment of the invention there is provided a hollow needle assembly comprising:

a) A needle constructed of one or more than one part, and comprising a hub with a blunt open end and a passage, and a shaft having a sharp open end and a lumen. The hub comprises a back end, which houses the blunt open end, and a front end, from which the shaft extends. The lumen is fluidly connected to the passage, and the needle further comprises a needle flow path defined along the lumen and the passage, beginning at the sharp open end of the shaft and terminating at the blunt open end of the hub.

b) A barrel constructed of one or more than one part, comprising an open anterior end through which a portion of the shaft of the needle passes, and an open posterior end through which a portion of the hub of the needle passes. The barrel further comprises an internal chamber for housing at least a portion of the needle.

The hub of the needle can move forward inside the barrel, for extending the sharp open end of the shaft of the needle for insertion into a vessel, e.g. a blood vessel, a catheter, or a capped tube. After use, the hub can move backwards inside the barrel for retracting the shaft of the needle into the barrel. The blunt open end of the needle can be fluidly connected to the inlet opening of a measurement apparatus, so that the blood can flow directly from a vessel, into the measurement apparatus, without the use of a syringe. The hollow needle assembly can also be used with a traditional syringe, as an alternative to traditional needles, in order to minimize the risk of needle-stick injury.

Other aspects and features of the present invention will become apparent, to those ordinarily skilled in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1A is a schematic drawing showing a top view of a needle for a hollow needle assembly according to a first embodiment of the invention;

FIG. 1B is a left side-view of the apparatus shown in FIG. 1A;

FIG. 1C is a right side-view of the apparatus shown in FIG. 1A;

FIG. 1D is a cross-sectional view through the apparatus shown in FIG. 1A along line D-D;

FIG. 1E is a perspective view of the apparatus shown in FIG. 1A;

FIG. 1F is detailed view of the detail F shown in FIG. 1E;

FIG. 2A is a schematic drawing showing a top view of a barrel for a hollow needle assembly according to a first embodiment of the invention;

FIG. 2B is a left side-view of the apparatus shown in FIG. 2A;

FIG. 2C is a cross-sectional view through the apparatus shown in FIG. 2A along line C-C;

FIG. 2D is a right side-view of the apparatus shown in FIG. 2A;

FIG. 2E is a cross-sectional view through the apparatus shown in FIG. 2A along line E-E;

FIG. 2F is a perspective view of the apparatus shown in FIG. 2A;

FIG. 3A is a schematic drawing showing a top view of a needle for a hollow needle assembly according to a second embodiment of the invention;

FIG. 3B is a left side-view of the apparatus shown in FIG. 3A;

FIG. 3C is a right side-view of the apparatus shown in FIG. 3A;

FIG. 3D is a cross-sectional view through the apparatus shown in FIG. 3A along line D-D;

FIG. 3E is a perspective view of the apparatus shown in FIG. 3A;

FIG. 3F is an alternative perspective view of the apparatus shown in FIG. 3A;

FIG. 4A is a schematic drawing showing a top view of a barrel for a hollow needle assembly according to a second embodiment of the invention;

FIG. 4B is a left side-view of the apparatus shown in FIG. 4A;

FIG. 4C is a cross-sectional view through the apparatus shown in FIG. 4A along line C-C;

FIG. 4D is a right side-view of the apparatus shown in FIG. 4A;

FIG. 4E is an alternative cross-sectional view through the apparatus shown in FIG. 4A along line E-E;

FIG. 4F is a perspective view of the apparatus shown in FIG. 4A;

FIG. 5A is a schematic drawing showing a top view of a needle and barrel assembled together with the needle concealed within the barrel, for a hollow needle assembly according to the second embodiment of the invention;

FIG. 5B is a left side-view of the apparatus shown in FIG. 5A;

FIG. 5C is a right side-view of the apparatus shown in FIG. 5A;

FIG. 5D is a cross-sectional view through the apparatus shown in FIG. 5A along line D-D;

FIG. 5E is a perspective view of the apparatus shown in FIG. 5A;

FIG. 5F is an alternative perspective view of the apparatus shown in FIG. 5A;

FIG. 6A is a schematic drawing showing a top view of the needle and barrel assembled together, with the needle extended outside the barrel, for a hollow needle assembly according to a third embodiment of the invention;

FIG. 6B is a cross-sectional view through the apparatus shown in FIG. 6A along line B-B;

FIG. 6C is an alternative cross-sectional view through the apparatus shown in FIG. 6A along line C-C;

FIG. 6D is a perspective view of the apparatus shown in FIG. 6A;

FIG. 7A is a schematic drawing showing a top view of a needle and barrel assembled together with the needle extended outside the barrel, for a hollow needle assembly according to a fourth embodiment of the invention;

FIG. 7B is a left side-view of the apparatus shown in FIG. 7A;

FIG. 7C is a right side-view of the apparatus shown in FIG. 7A;

FIG. 7D is a cross-sectional view through the apparatus shown in FIG. 7A along line D-D;

FIG. 7E is detailed view of the detail E shown in FIG. 7D;

FIG. 8A is a schematic drawing showing a top view of the needle and barrel assembly shown in FIGS. 7A-E, with the needle concealed inside the barrel, and with an optional safety cap on for a hollow needle assembly according to the fourth embodiment of the invention;

FIG. 8B is a left side-view of the apparatus shown in FIG. 8A;

FIG. 8C is a right side-view of the apparatus shown in FIG. 8A;

FIG. 8D is a cross-sectional view through the apparatus shown in FIG. 8A along line D-D;

FIG. 9A is a schematic drawing showing a top view of a needle and barrel assembled together, with the needle concealed inside the barrel, with a measurement apparatus 600a attached, and an optional safety cap on for a hollow needle assembly according to the fourth embodiment of the invention;

FIG. 9B is a cross-sectional view through the apparatus shown in FIG. 9A along line B-B;

FIG. 9C is a perspective view of the apparatus shown in FIG. 9A;

FIG. 10A is a schematic drawing showing a top view of a needle also comprising a measurement apparatus like 600a shown in FIGS. 9A-C, for a hollow needle assembly according to a fifth embodiment of the invention;

FIG. 10B is a cross-sectional view through the apparatus shown in FIG. 10A along line B-B;

FIG. 10C is a perspective view of the apparatus shown in FIG. 10A;

FIGS. 11A-G are schematic drawings showing details of the measurement apparatus 600a shown in FIGS. 9A-C;

FIG. 12A is a schematic drawing showing a top view of a needle also comprising a measurement apparatus 600b, for a hollow needle assembly according to a sixth embodiment of the invention;

FIG. 12B is a cross-sectional view through the apparatus shown in FIG. 12A along line B-B;

FIG. 12C is a perspective view of the apparatus shown in FIG. 12A;

FIGS. 13A-E are schematic drawings showing details of the measurement apparatus 600b shown in FIGS. 12A-C;

FIGS. 14A-G are schematic drawings showing details of the hollow fiber bundle 660 shown in FIGS. 13A-E;

FIGS. 15A-C are schematic drawings showing details of a measurement apparatus 600c that can be used with the needle of the first embodiment of the invention, as shown in FIGS. 1A-F;

FIG. 16A is a schematic drawing showing a top view of the needle and barrel assembled together, with the needle extended outside the barrel, for a hollow needle assembly according to a seventh embodiment of the invention;

FIG. 16B is a cross-sectional view through the apparatus shown in FIG. 16A along line B-B;

FIG. 16C is a perspective view of the apparatus shown in FIG. 16A;

FIG. 16D is a detailed view of the detail D shown in FIG. 16B;

FIG. 17A is a schematic drawing showing a front view of the needle and barrel assembled together, with the needle extended outside the barrel, for a hollow needle assembly according to an eight embodiment of the invention;

FIG. 17B is a schematic drawing showing a top view of the needle and barrel assembly shown in FIG. 17A;

FIG. 17C is a perspective view of the apparatus shown in FIG. 17A;

FIG. 18A is a schematic drawing showing a top view of the needle and barrel assembled together, with the needle extended outside the barrel, for a hollow needle assembly according to a ninth embodiment of the invention;

FIG. 18B is a right side-view of the apparatus shown in FIG. 18A;

FIG. 18C is a cross-sectional view through the apparatus shown in FIG. 18A along line C-C;

FIG. 19A is a schematic drawing showing the ninth embodiment of a hollow needle and barrel assembled together, with a syringe in position prior to engagement with the needle;

FIG. 19B is a cross-sectional view through the apparatus shown in FIG. 19A along line B-B; and

FIG. 19C is a perspective view of the apparatus shown in FIG. 19A.

FIG. 20A is a schematic drawing showing the ninth embodiment of a hollow needle and barrel assembled together, with an adaptor in position prior to converting the blunt open end of the needle, from a female into a male configuration;

FIG. 20B is a cross-sectional view through the apparatus shown in FIG. 20A along line B-B; and

FIG. 20C is a perspective view of the apparatus shown in FIG. 20A.

### DETAILED DESCRIPTION OF PREFERRED ASPECTS OF THE INVENTION

Some embodiments of the invention provide a hollow needle assembly that is suitable for collection of a sample directly from a vessel, for example without limitations, a blood vessel of a patient into the measurement apparatus. Some embodiments of the invention provide an apparatus that is suitable for both the collection and measurement of a blood sample; and some embodiment of the invention provide one apparatus that is suitable for the collection of a blood sample, the extraction of plasma from the blood (sometimes referred to as whole blood, to distinguish blood from serum and plasma), and the measurement of both the whole blood and the plasma extracted from the whole blood. Currently a needle and syringe is required to collect the blood, and subsequently the blood is injected into the measurement apparatus after removing the needle from the syringe. The transfer of blood from a syringe to a measurement apparatus causes delays in testing, and an anticoagulant is required when blood is not tested within the first few minutes of collection. Moreover, handling the needle increases the risk of infection due to injury by the needle and subsequent infection by blood-borne pathogens, and in general, handling the blood in open vessels increases the risk of contamination by blood-borne pathogens. A further complication caused by the transfer of blood from a syringe to a measurement apparatus is contamination with air. Although blood is the fluid used to illustrate the function of the apparatus, those skilled in the art will appreciate that the present invention can also be used, for example without limitation, to transfer fluid from a plastic or rubber bag, vacutainer tube, or tubing, to a measurement apparatus. Once a blood sample is drawn into a measurement apparatus, the blood sample can be analyzed without delay, and without having to transfer any portion of the blood sample into another vessel.

Some embodiments of the invention provide alternatives to traditional needles and are suitable for collecting blood into a syringe, with minimal risks of finger-stick injury. Traditional needles and syringes expose the user to finger-stick injuries during removal of the needle from the syringe, or during recapping of the needle. Current medical practice strongly advises against recapping needles in syringes, due to the risk of injury by the needle contaminated with blood, which may contain hazardous pathogens. In accordance with several embodiments of the invention, recapping or removing the needle is not required, and examples of specific embodiments are shown, where the needle can be retracted into a barrel, and then as optionally, the anterior end of the barrel can be capped, as a further safeguard against finger-stick injury.

The main parts of the present invention are a needle and a barrel, with an optional safety cap, which engages onto the open anterior end, an optional locking cap for locking the needle in position, and an optional spring for automatic needle retraction after the locking cap is loosened. Some embodiments of the invention use a stud and slot mechanism for keeping the studded section of the hub within the slot of the barrel. Those skilled in the art will appreciate that the stud could be a separate part, which is screwed into the hub after assembly of the needle and barrel. In some embodiments of the invention, the measurement apparatus is integrated with the hub or the needle. In these embodiments, the vent of the apparatus becomes the blunt open end of the needle

Several embodiments of the invention are described in details, in order to describe the present invention. The common features in the different embodiments are a needle with a flow path that begins at a sharp open end in the shaft of the needle and terminates at a blunt open end in the hub of the needle, and a mobile barrel that facilitates extension and retraction of the sharp open end of the needle. In some embodiments, the barrel has an open anterior end, which acts as a first annular stop, and an open posterior end, which acts as a second annular stop. Moreover, in some embodiments, a screw cap functions as the second annular stop. Those skilled in the art will appreciate that in some embodiments, annular stops are not essential.

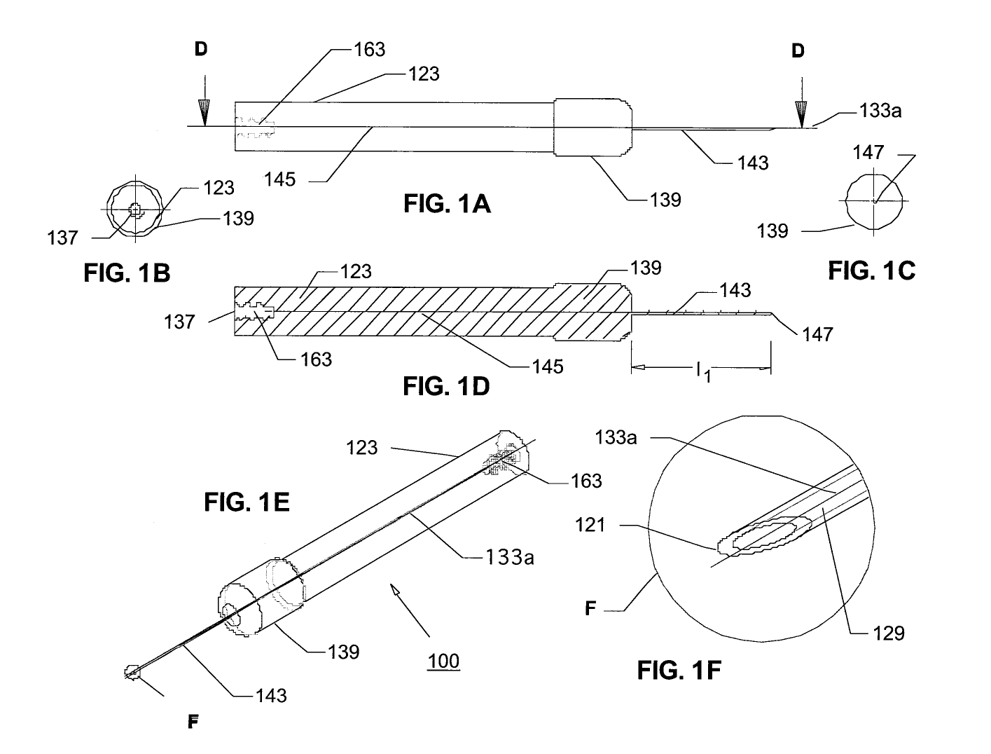

Referring to FIG. 1A, shown is a schematic drawing illustrating a top view of a needle 100 for a hollow needle assembly according to a first embodiment of the invention; FIG. 1B illustrates a left side-view of the apparatus shown in FIG. 1A; FIG. 1C illustrates a right side-view of the apparatus shown in FIG. 1A; FIG. 1D illustrates a cross-sectional view through the apparatus shown in FIG. 1A along line D-D; FIG. 1E illustrates a perspective view of the apparatus shown in FIG. 1A; and FIG. 1F illustrates a detailed view of the detail F shown in FIG. 1E.

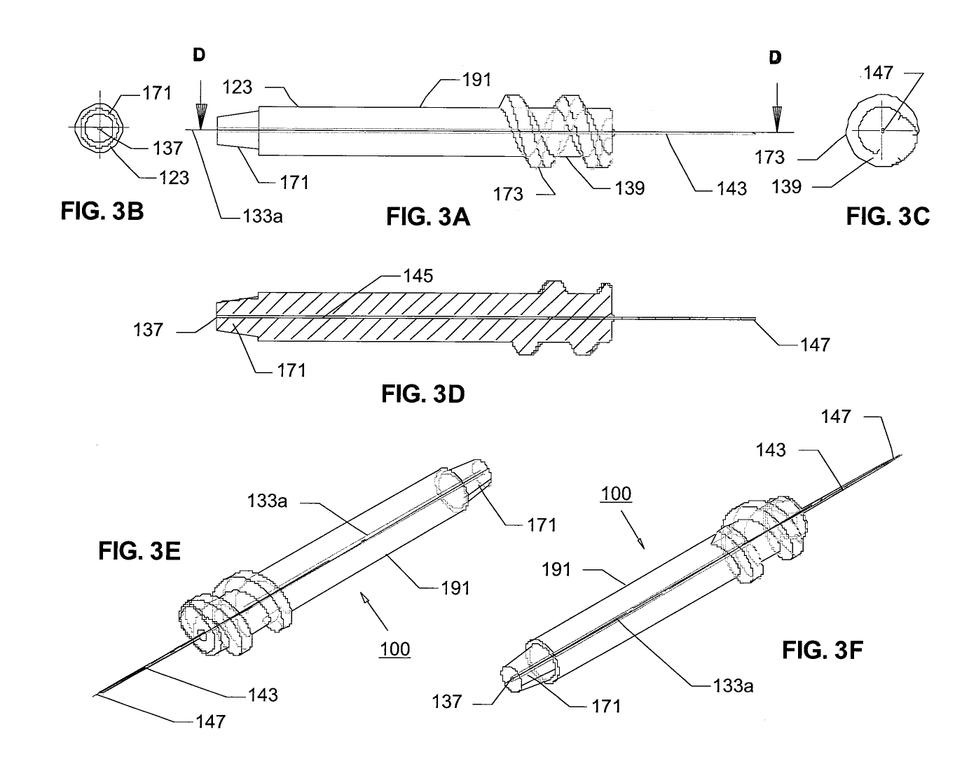

Still referring to FIG. 1, the needle 100 comprises a shaft 143 and a hub (shown as 191 in FIGS. 3A, E and F) with a front end 139 and a back end 123. The shaft 143 has a sharp open end 147 and a second end, which is mounted in the passage 145 of the hub. A detailed view of the sharp open end 147 (detail F in FIG. 1E) is shown in FIG. 1F. The sharp open end 147 is usually the beveled end of the shaft that is usually a hollow metal tube. It should be understood that the sharp open end 147 could be configured differently from a bevel, and that a bevel should not limit the scope of the invention in any way. The hollow portion of the shaft is also referred to as the lumen 129. The bevel provides a point 121 for piercing a vessel, for example without limitation, a blood vessel. Also shown in FIG. 1F is the central axis 133a, which runs through the center of the shaft 143, along its length. The length of the shaft 143 outside the hub 191 is shown to have a length dimension I₁. The section of the shaft 143 mounted inside the hub is not shown. The front end of the hub is shown as 139, and the back end of the hub is shown as 123. It should be understood that the front end refers to a general area of the hub, and does not specifically identify any point or local area. Similarly, it should be understood that the back end refers to a general area of the hub, and does not specifically identify any point or local area. The passage 145 of the hub is fluidly connected to the lumen 129 of the shaft, and a first flow path is defined by the sharp open end 147, which leads into the lumen 129, which leads into the passage 145 of the hub, and terminates at a blunt open end 137. The blunt open end 137 is located at the back end of the hub. The hub could comprise other features, and some features are described with the descriptions of other embodiments of the invention.

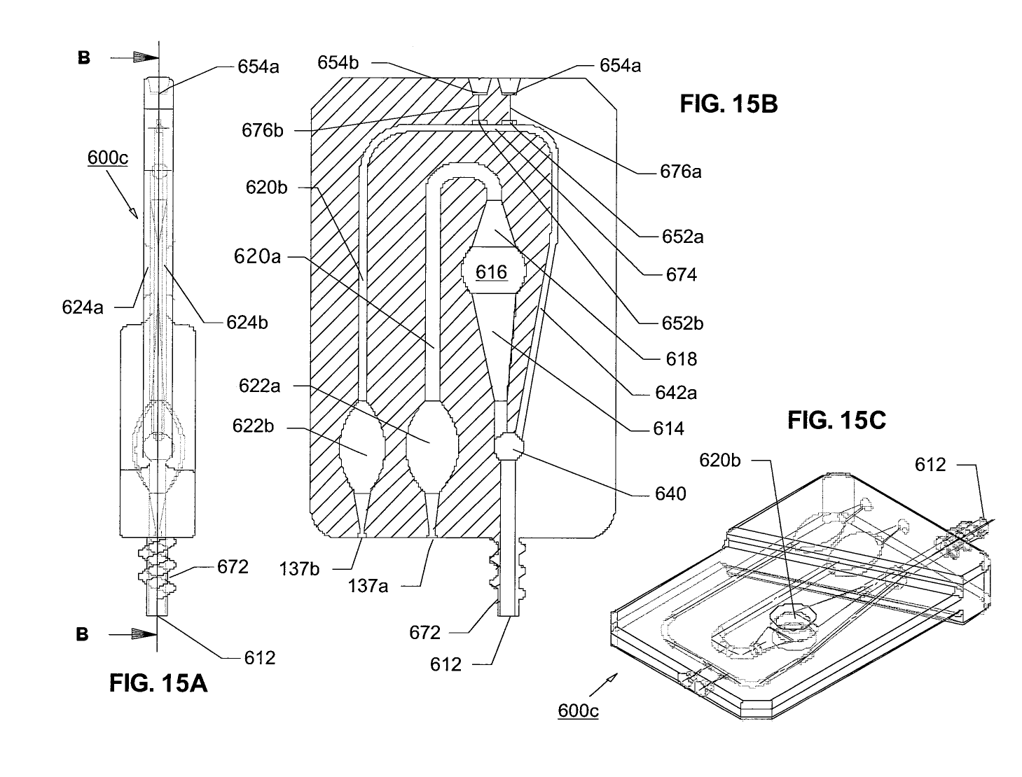

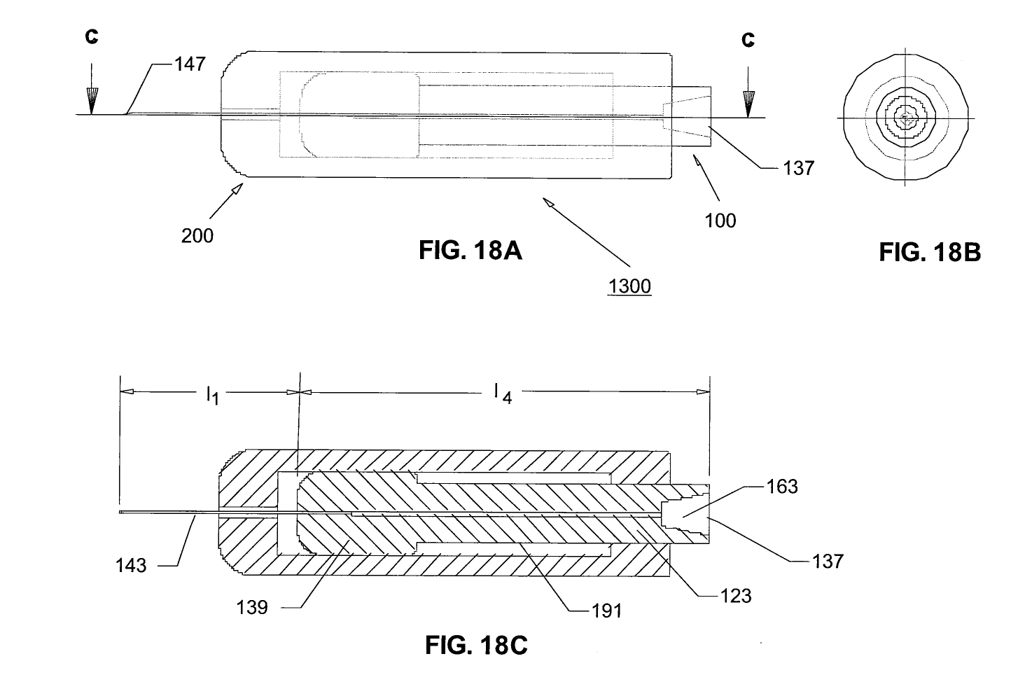

Still referring to FIG. 1, the back end of the hub 123 also houses a female receptor 163 with internal threads, for receiving a measurement apparatus through the blunt open end 137. An example of a measurement apparatus is illustrated in FIGS. 15A-C as 600c. Mating external threads are shown in tubing 672 of FIGS. 15A-B, for securing or locking the hollow needle assembly to the measurement apparatus 600c. In another aspect of the invention, the female receptor 163 is configured to accept the male end of a syringe, illustrated in FIGS. 18A-C, 19A-C and 20A-C. The assembly of the needle and barrel in a hollow needle assembly is illustrated in several other embodiments of the invention.

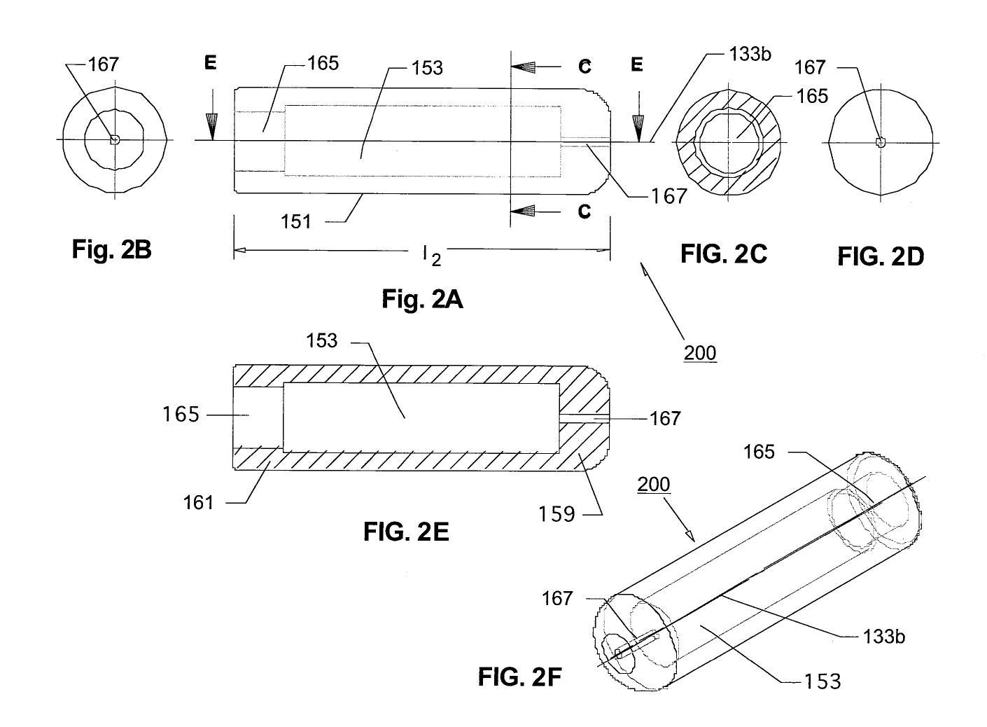

Referring to FIG. 2A, shown is a schematic drawing illustrating a top view of a barrel 200 for a hollow needle assembly according to the first embodiment of the invention; FIG. 2B illustrates a left side-view of the apparatus shown in FIG. 2A; FIG. 2C illustrates a cross-sectional view through the apparatus shown in FIG. 2A along line C-C; FIG. 2D illustrates a right side-view of the apparatus shown in FIG. 2A; FIG. 2E illustrates an alternative cross-sectional view through the apparatus shown in FIG. 2A along line E-E; and FIG. 2F illustrates a perspective view of the apparatus shown in FIG. 2A. The barrel 200 shown in FIG. 2 must be combined with the needle 100 shown in FIGS. 1A-F, to provide the first embodiment of a hollow needle assembly. Other embodiments of hollow needle assemblies are illustrated later. Also illustrated in FIGS. 2A, B and D-F is an opening 167 for the needle shaft 143 (FIGS. 1A, B and E) in the open anterior end 159 of the barrel 200, an opening 165 for the back end of the hub 123 (FIGS. 1A, B and E) in the open posterior end 161 of the barrel 200, and an axis 133b which runs through the center of the barrel, along the length dimension of the barrel, shown as I₂. In order for the barrel to conceal the sharp end of the shaft, 147 (FIG. 1), I₂ must be greater than I₁. The barrel 200 comprises an internal chamber 153 for housing the front end 139 of the hub. In the specific embodiments shown later, the central axis 133a of the needle and axis 133b of the barrel are shown to be coaxial, but the axes could also be parallel without being coaxial, for example, if the outer design of the barrel is not cylindrical.

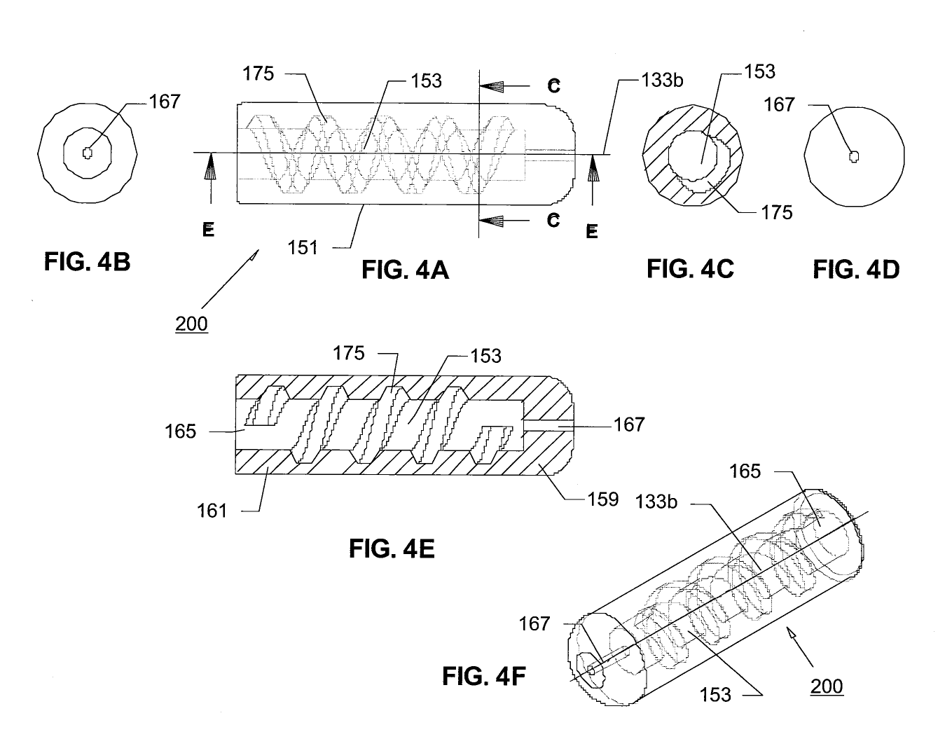

Referring to FIG. 3A, shown is a schematic drawing illustrating a top view of a needle for a hollow needle assembly according to a second embodiment of the invention; FIG. 3B illustrates a left side-view of the apparatus shown in FIG. 3A; FIG. 3C illustrates a right side-view of the apparatus shown in FIG. 3A; FIG. 3D illustrates a cross-sectional view through the apparatus shown in FIG. 3A along line D-D; FIG. 3E illustrates a perspective view of the apparatus shown in FIG. 3A; and FIG. 3F illustrates an alternative perspective view of the apparatus shown in FIG. 3A. The apparatus 100 illustrated in FIG. 3 is similar to the apparatus 100 illustrated in FIG. 1, and accordingly, elements common to both share common reference numerals. The primary differences, illustrated in FIG. 3, are that the back end 139 of the hub 191 contains external threads 173 for mating with internal threads 175 in a complementary barrel 200 shown in FIGS. 4A, C and E, and the back end of the hub 123 houses the blunt open end 137 in a tapered projection 171, which houses the blunt open end 137, wherein the tapered projection resembles the male end of a syringe. Those skilled in the art will appreciate that other suitable mating ends can be used, for example without limitations, internal and external threads, and Luer lock mechanisms, and are considered to be within the scope of the present invention.

Referring to FIG. 4A, shown is a schematic drawing illustrating a top view of a barrel 200 for a hollow needle assembly according to the second embodiment of the invention; FIG. 4B illustrates a left side-view of the apparatus shown in FIG. 4A; FIG. 4C illustrates a cross-sectional view through the apparatus shown in FIG. 4A along line C-C; FIG. 4D illustrates a right side-view of the apparatus shown in FIG. 4A; FIG. 4E illustrates an alternative cross-sectional view through the apparatus shown in FIG. 4A along line E-E; and FIG. 4F illustrates a perspective view of the apparatus shown in FIG. 4A. The apparatus 200 illustrated in FIG. 4 is similar to the apparatus 200 illustrated in FIG. 2, and accordingly, elements common to both share common reference numerals. The primary difference, illustrated in FIG. 4, is the internal threads 175. The threads 175 as shown in FIG. 4, do not run continuously throughout the length of the barrel, and prevents the hub from moving beyond the threaded area of the barrel 200, even if the opening 167 was larger than the opening 165. It should be understood that although in the preferred embodiment the opening as illustrated in 167 is small, yet large enough for the shaft of the needle to penetrate, the size of the opening 167 should not limit the scope of the invention in any way.

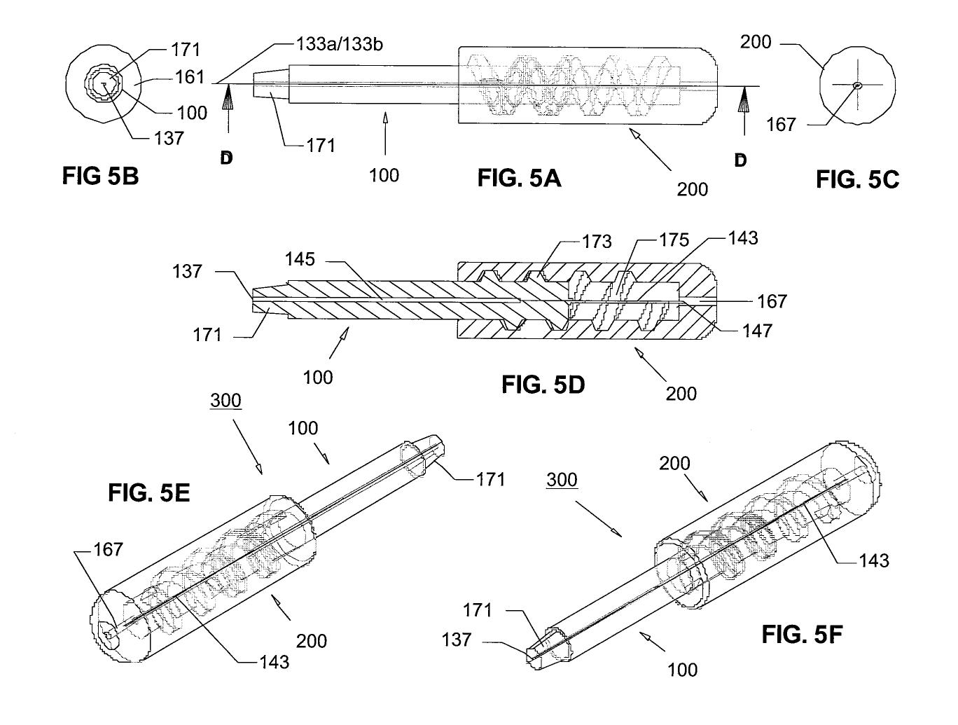

Referring to FIG. 5A, shown is a schematic drawing illustrating a top view of a needle and barrel assembly 300 with the shaft 143 of the needle 100 concealed within the barrel 200 according to the second embodiment of the invention; FIG. 5B illustrates a left side-view of the apparatus shown in FIG. 5A; FIG. 5C illustrates a right side-view of the apparatus shown in FIG. 5A; FIG. 5D illustrates a cross-sectional view through the apparatus shown in FIG. 5A along line D-D; FIG. 5E illustrates a perspective view of the apparatus 300 shown in FIG. 5A; and FIG. 5F illustrates an alternative perspective view of the apparatus 300 shown in FIG. 5A. The apparatus 300 illustrated in FIGS. 5A-F is an assembly of the needle 100 illustrated in FIG. 3, and the barrel 200 illustrated in FIG. 4, and accordingly, elements common to these share common reference numerals.

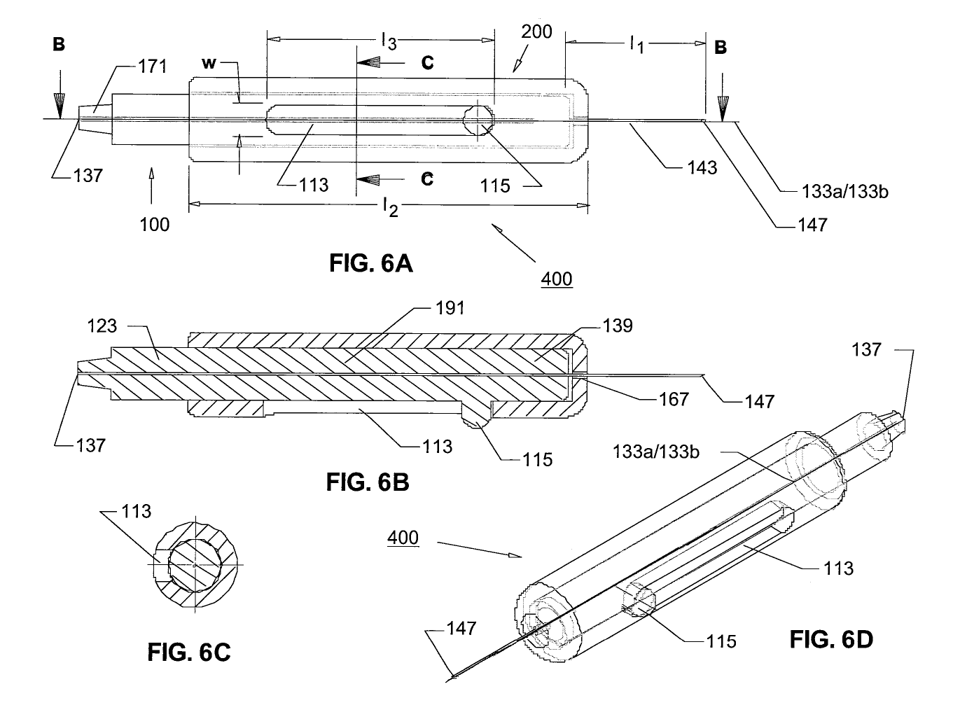

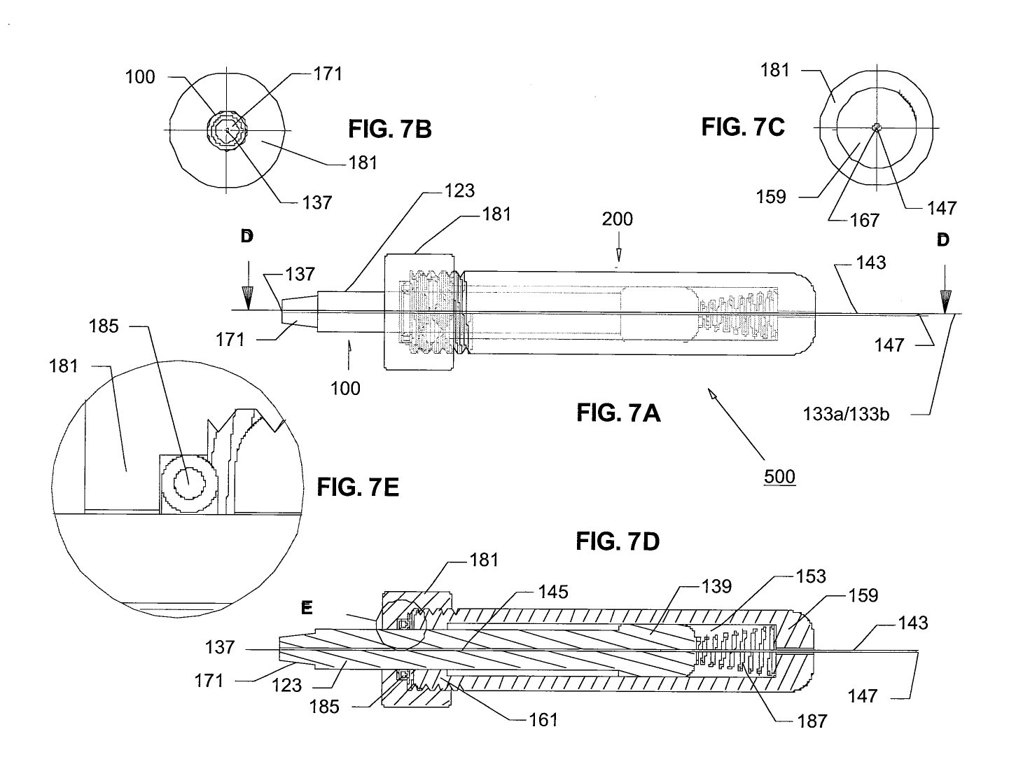

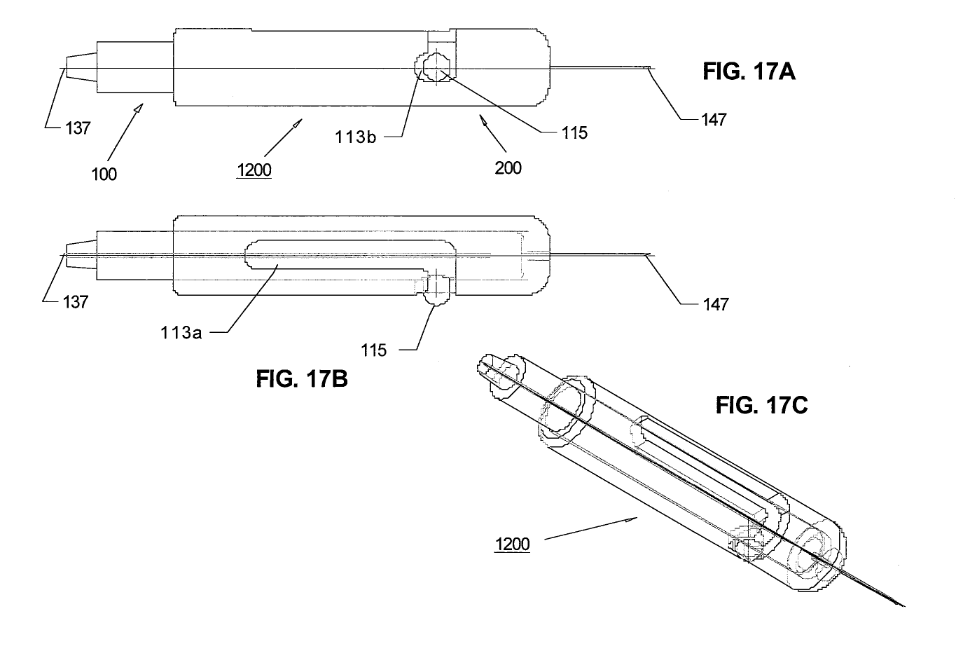

Referring to FIG. 6A, shown is a schematic drawing illustrating a top view of the needle and barrel assembly 400, with the needle 100 extended outside the barrel 200, for a hollow needle assembly according to a third embodiment of the invention; FIG. 6B illustrates a cross-sectional view through the apparatus shown in FIG. 6A along line B-B; FIG. 6C illustrates an alternative cross-sectional view through the apparatus shown in FIG. 6A along line C-C; and FIG. 6D illustrates a perspective view of the apparatus shown in FIG. 6A. The apparatus 400 illustrated in FIG. 6 is an assembly of a modified needle 100 illustrated in FIGS. 1A-F, and modified barrel 200 illustrated in FIGS. 2A-F, and accordingly, elements common to these share common reference numerals. The primary differences illustrated in FIGS. 6A-D are: in the needle 100, the external diameter of the hub 191 is uniform throughout most of the hub, the blunt open end 137 is housed in a tapered projection 171, which resembles the male end of a syringe, and a stud 115 projects from the hub, at a location around the front end 139 of the hub 191; in the barrel 200, the internal diameter of the internal chamber (shown in FIGS. 2A, E and F as 153) is uniform throughout the length I₂, except for the anterior end housing the opening 167; a slot 113 is cut through the wall of the barrel for a length I₃ and having a width w, wherein I₃ is at least slightly longer than the length of the shaft 143 shown as I₁. The internal diameter of the internal chamber 153 is approximately equal to the external diameter of the hub, in order for the needle 100 to slide smoothly inside the barrel 200, for extending and retracting the sharp open end 147 of the shaft 143. The stud 115 fits into the slot 113, with the stud slightly extended beyond the outer diameter of the barrel 200, in order to facilitate the smooth sliding motion of the needle 100 inside the barrel 200, by using pressing a finger against the stud 115. The width of the slot w is slightly larger than the diameter of the stud, in order for the slot 113 to act as a track for the stud 115, with little friction. The stud 115 can only move along the length I₃ of the slot 113, and helps to keep the needle inside the barrel. A locking cap as described as 181 in FIGS. 7A-D is not essential because the user could lock the needle in a position during use, by pressing a finger against the stud 115. As an alternative to this third embodiment of the invention, shown is an eight embodiment of the invention, illustrated in FIGS. 17A-C. The difference is the slot 113 shown in FIG. 6A is replaced with a slot 113a, with a hooked end 113b. The hooked end 113b is used for securing the stud 115, so that the needle cannot move relative to the barrel during insertion of the sharp end of the needle into a blood vessel, and applying pressure on the stud 115 is not necessary. In the illustrations of the third (FIGS. 6A-D) and eight embodiments (FIGS. 17A-C), there is no annular stop at the posterior end of the barrel, and the annular stop at the anterior end of the barrel (the part of the anterior end that houses the opening 167) is not essential as long as the sharp end 147 of the needle 100 is retracted into the barrel 200.

Referring to FIG. 7A, shown is a schematic drawing illustrating a top view of a needle and barrel assembly 500 with the sharp end 147 of the needle shaft 143 extended outside the barrel 200, according to a fourth embodiment of the invention; FIG. 7B illustrates a left side-view of the apparatus shown in FIG. 7A; FIG. 7C illustrates a right side-view of the apparatus shown in FIG. 7A; FIG. 7D illustrates a cross-sectional view through the apparatus shown in FIG. 7A along line D-D; and FIG. 7E illustrates a detailed view of the detail E shown in FIG. 7D. The needle 100 of apparatus 500 illustrated in FIGS. 7A-D is similar to the needle 100 illustrated in FIG. 1, and the barrel 200 of apparatus 500 illustrated in FIGS. 7A-D is similar to the barrel 200 illustrated in FIG. 2, and accordingly, elements common to them share common reference numerals. The primary differences, illustrated in FIGS. 7A-D are: a locking cap 181; external threads at the open posterior end 161 of the barrel 200, and a spring 187; the locking cap 181 is fitted with a flexible member 185 at the juncture of the locking cap 181 and the open posterior end 161 of the barrel; the locking cap 181 has internal threads that mate with the external threads at the open posterior end 161 of the barrel 200.

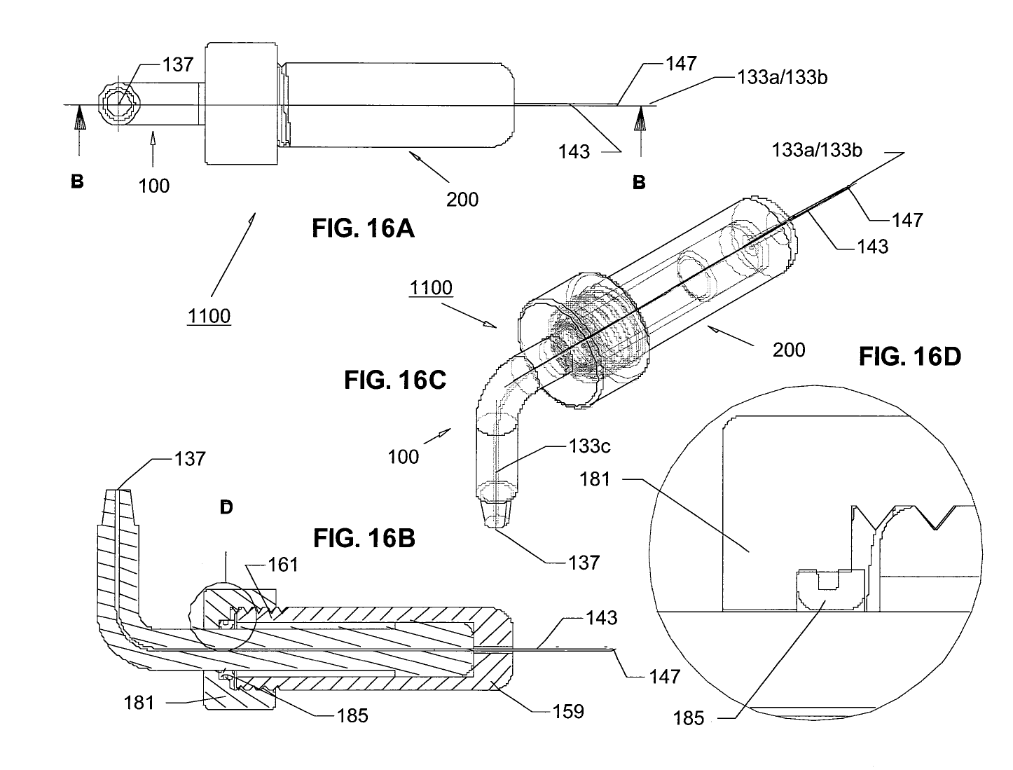

Still referring to FIGS. 7A-D, the spring 187 is located within the internal chamber 153, between the open anterior end 159 of the barrel, and the front end 139 of the hub. The flexible member 185 is a hollow O-ring preferably made from plastic or rubber, and expands towards the axes 133a and 133b when the locking cap 181 is tightened, causing the flexible member 185 to press against the hub. As the flexible member 185 presses against the hub, the needle becomes locked in position. Although threads are a preferred means of operating the locking cap 181, those skilled in the art will appreciate that a locking cap could also operate by frictional engagement of a locking cap similar to that of the apparatus 500 illustrated in FIGS. 7A-D but without threads, to the open posterior end 161 of the barrel 200 without threads. A second embodiment of a flexible member 185 (an O-ring with a C-shaped cross-sectional area) is shown in FIGS. 16b and D, and it should be understood that these are just non-limiting examples of means used to lock the needle in position. Those skilled in the art will appreciate that other means of locking the needle in position exist, and are considered to be within the scope of the present invention.

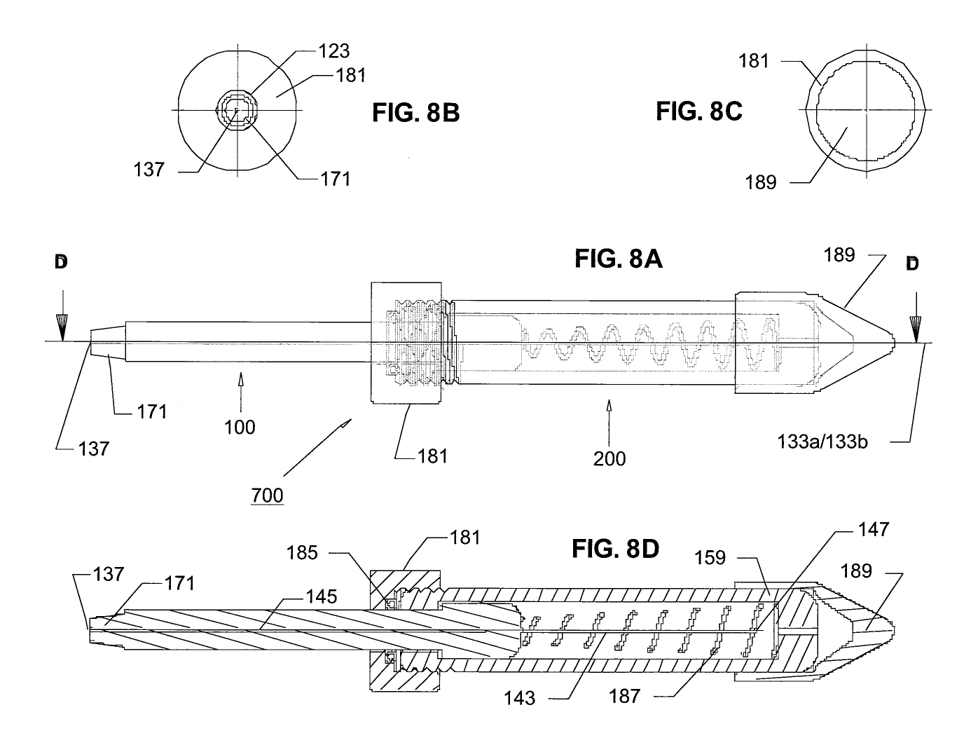

Referring to FIG. 8A, shown is a schematic drawing illustrating a top view of the needle and barrel assembly 700, as shown in FIGS. 7A-D, with the sharp end 147 of the shaft 143 concealed inside the barrel 200, and with an optional safety cap 189 engaged, according to the fourth embodiment of the invention; FIG. 8B illustrates a left side-view of the apparatus shown in FIG. 8A; FIG. 8C illustrates a right side-view of the apparatus shown in FIG. 8A; and FIG. 8D illustrates a cross-sectional view through the apparatus shown in FIG. 8A along line D-D. The apparatus 700 illustrated in FIGS. 8A-D is similar to the apparatus 500 illustrated in FIGS. 7A-D, and accordingly, elements common to both share common reference numerals. The primary differences, illustrated in FIGS. 8A and D is the safety cap 189 fitted over the open anterior end 159 of the barrel 200, to further protect the user from needle-stick injury.

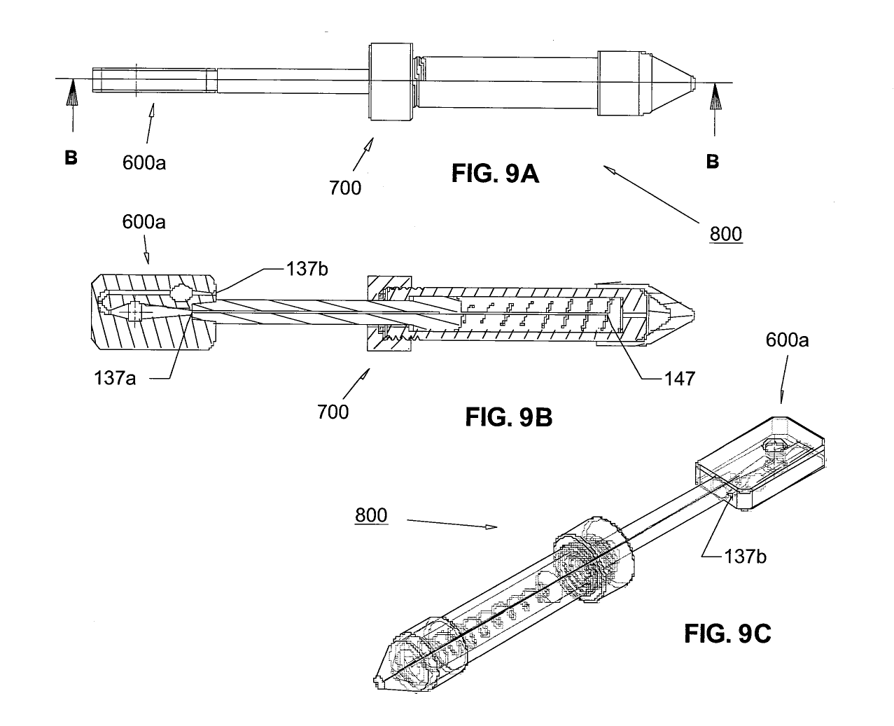

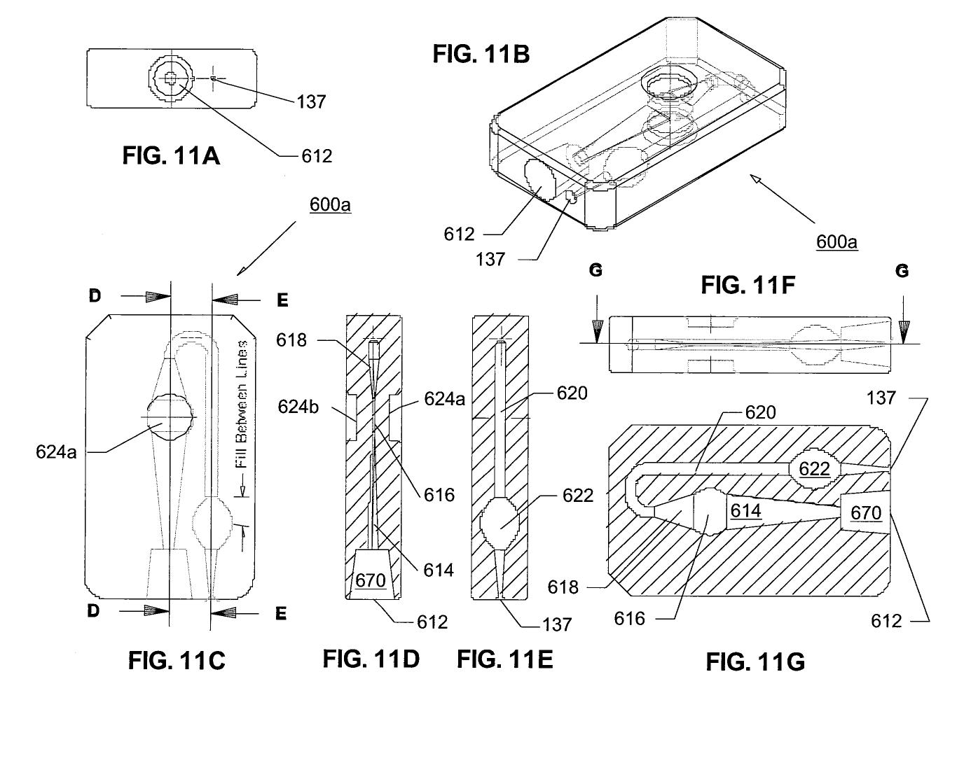

Referring to FIG. 9A, shown is a schematic drawing of an apparatus 800, illustrating a top view of a needle and barrel assembly 700 shown in FIGS. 8A-D, with a measurement apparatus 600a attached, according to the fourth embodiment of the invention; FIG. 9B illustrates a cross-sectional view through the apparatus shown in FIG. 9A along line B-B; and FIG. 9C illustrates a perspective view of the apparatus shown in FIG. 9A. Details of the measurement apparatus 600a are illustrated in FIGS. 11A-G. The blunt open end of the hollow needle assembly 700 is shown as 137a. When apparatus 600a and apparatus 700 are fluidly connected, the new blunt open end of the extended fluid path is shown as the vent 137b of the measurement apparatus 600a.

Use of the hollow needle assembly and measurement apparatus shown collectively in FIGS. 7A-D, FIGS. 8A-D, FIGS. 9A-C, and FIGS. 11A-G, will be described for filling the apparatus 600a with blood from a blood vessel, as a non-limiting example. It will be appreciated by those skilled in the art, that the steps described below may be slightly different for other embodiments of the hollow needle assembly. Before use, the hollow needle assembly 700 will look like the illustration shown in FIG. 8A. An example of the use of the embodiments illustrated, requires the following steps:
1. Insert the blunt open end 171 of the needle 100 securely into the inlet chamber 670 of the measurement apparatus 600a. The hollow needle assembly 700 attached to the apparatus 600a will look like the illustration 800 shown in FIGS. 9A-C.
2. Remove the optional safety cap 189.
3. Loosen the locking cap 181 and carefully extend the shaft of the needle by pushing the hub of the needle 100 against the spring 187. Tighten the looking cap to maintain the needle in the extended position. The hollow needle assembly 700 (the apparatus 600a is not shown) will now look like the illustration 500 shown in FIG. 7A.
4. Carefully insert the sharp open end 147 of the needle into the blood vessel, following standard procedures know by doctors and phlebotomists.
5. Allow the blood to flow into the measurement apparatus 600a, via the needle 100, until the blood is between the two “fill between lines” shown in FIG. 11C. Blood will flow according to the blood pressure within the blood vessel. In the case of an artery, where the blood pressure is higher than the pressure in a vein, more case must be taken. The capillary break 622 shown in FIGS. 11E and G is used as a buffer zone to prevent blood from escaping through the vent 137. In the case of a vein, application of a tourniquet may be necessary. Capillary action may also help draw blood into the apparatus, depending on the internal dimensions of the flow path, and the hydrophilic properties of the internal surfaces of the flow path.
6. Carefully withdraw the needle from the blood vessel according to standard practice.
7. Slowly loosen the locking cap 181, allowing the force of the spring 187 to retract the sharp end 147 of the needle 100 into the barrel 200.
8. Tighten the locking cap 181 to keep the needle inside the barrel. Optionally, the safety cap 189 could be replaced.

An example of a method of filling a syringe with blood using the hollow needle assembly, includes the following steps:
1. Engaging the blunt open end of the hollow needle assembly, to the male end of a syringe;
2. Extending the shaft of the needle of the hollow needle assembly;
3. Piercing a vessel with the sharp open end of the needle of the hollow needle assembly;
4. Allowing the blood to flow into the syringe, via the needle;
5. Withdrawing the needle from the vessel; and
6. Retracting the needle into the barrel.
Those skilled in the art will appreciate that the hollow needle assembly could be used with other fluids, for example without limitations, dairy products; and other vessels, for example without limitations, bags, tubings, and capped tubes.

An example of a method of filling a measurement apparatus comprising at least one flow path beginning at an inlet opening and terminating at a vent, with blood from a vessel, includes the following steps:
1. Engaging the blunt open end of the hollow needle assembly, to the inlet opening of the measurement apparatus;
2. Extending the shaft of the needle of the hollow needle assembly;
3. Piercing the vessel with the sharp open end of the needle of the hollow needle assembly;
4. Allowing the blood to flow into the measurement apparatus, via the needle;
5. Withdrawing the needle from the vessel; and
6. Retracting the needle into the barrel.
Those skilled in the art will appreciate that the hollow needle assembly could be used with other fluids, for example without limitations, dairy products; and other vessels, for example without limitations, bags, tubings, and capped tubes.

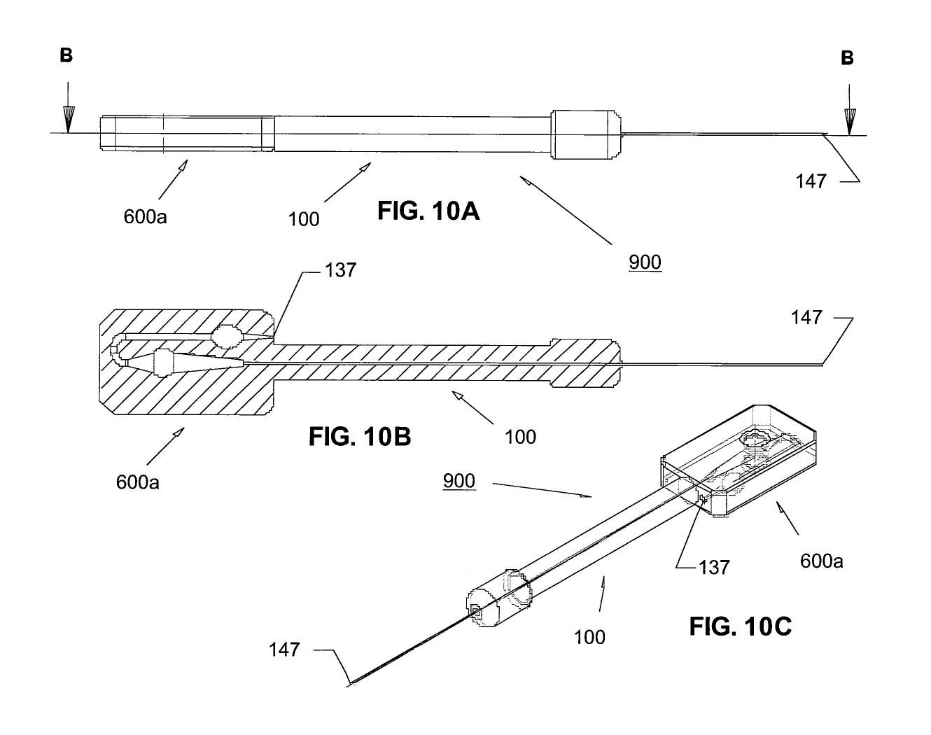

As a non-limiting example illustrated in FIGS. 10A-C, the needle 100 and the measurement apparatus 600a could be integrated.

Referring to FIG. 10A, shown is a schematic drawing of an apparatus 900, illustrating a top view of a needle 100, wherein the measurement apparatus 600a (illustrated in FIGS. 11A-G) is an integral part of the hub of the needle 100, according to a fifth embodiment of the invention; FIG. 10B illustrates a cross-sectional view through the apparatus shown in FIG. 10A along line B-B; and FIG. 10C illustrates a perspective view of the apparatus shown in FIG. 10A. The only blunt open end is the vent 137 of the measurement apparatus 600a. Moreover, in the fifth embodiment of the invention, a single flow path is defined from the sharp open end 147, to the blunt open end 137. The needle 100 and measurement apparatus 600a together form a needle with a larger hub, and with the flow path of the measurement apparatus 600a integrated with the flow path of the hollow needle assembly.

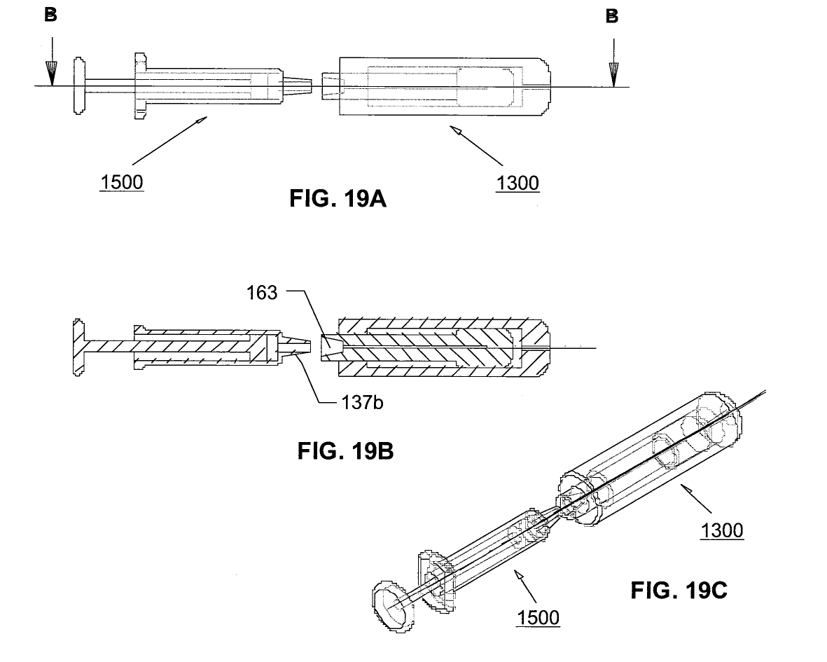

Referring to FIGS. 11A-G, shown are schematic drawings providing details of the measurement apparatus 600a illustrated in FIGS. 9A-C and FIGS. 10A-C. The measurement technology includes spectroscopy with the optional use of one or more than one reagent. Referring to FIG. 11A, shown is schematic drawing of a front view of the measurement apparatus 600a illustrated in FIGS. 9A-C and FIGS. 10A-C, showing the sample inlet opening 612 and the vent 137. Referring to FIG. 11B, shown is a perspective view of the measurement apparatus 600a. Referring to FIG. 11C, shown is a schematic drawing of a top view of the apparatus shown in FIG. 11A, with a wall-portion 624a of the optical chamber 616, and two guide lines for filling the apparatus with blood. Referring to FIG. 11D, shown is a cross-sectional view of the apparatus illustrated in FIG. 11C along line D-D. Still referring to FIG. 11D, shown is a schematic drawing of the inlet opening 612, the inlet chamber 670, which can accept the outlet 171 of a needle (for example, 171 shown in FIGS. 3A, D, E and F), the inlet transition chamber 614, the optical chamber 616, the overflow chamber 618, the optical chamber wall-portions 624a and 624b. Referring to FIG. 11E, shown is a cross-sectional view through the apparatus 600a illustrated in FIG. 11C along line E-E, showing the outflow 620, the capillary break 622, and the vent 137. Referring to FIG. 11F, shown is a left side-view of the apparatus 600a illustrated in FIG. 11C. Referring to FIG. 11G, shown is an alternative cross-sectional view through the apparatus 600a illustrated in FIG. 11F along line G-G, showing the complete flow path, beginning at the sample inlet opening 612, and terminating at the vent 137, with the inlet chamber 670, the inlet transition chamber 614, the optical chamber 616, the overflow chamber 618, the outflow chamber 620, and the capillary break 622 fluidly connected in series. Those skilled in the art will appreciate that different designs of cartridges can be used as the measurement apparatus, and for the sake of brevity, measurement apparatuses will not be discussed in great details. Moreover, other uses of the present invention will be illustrated in FIGS. 19A-C and 20A-C.

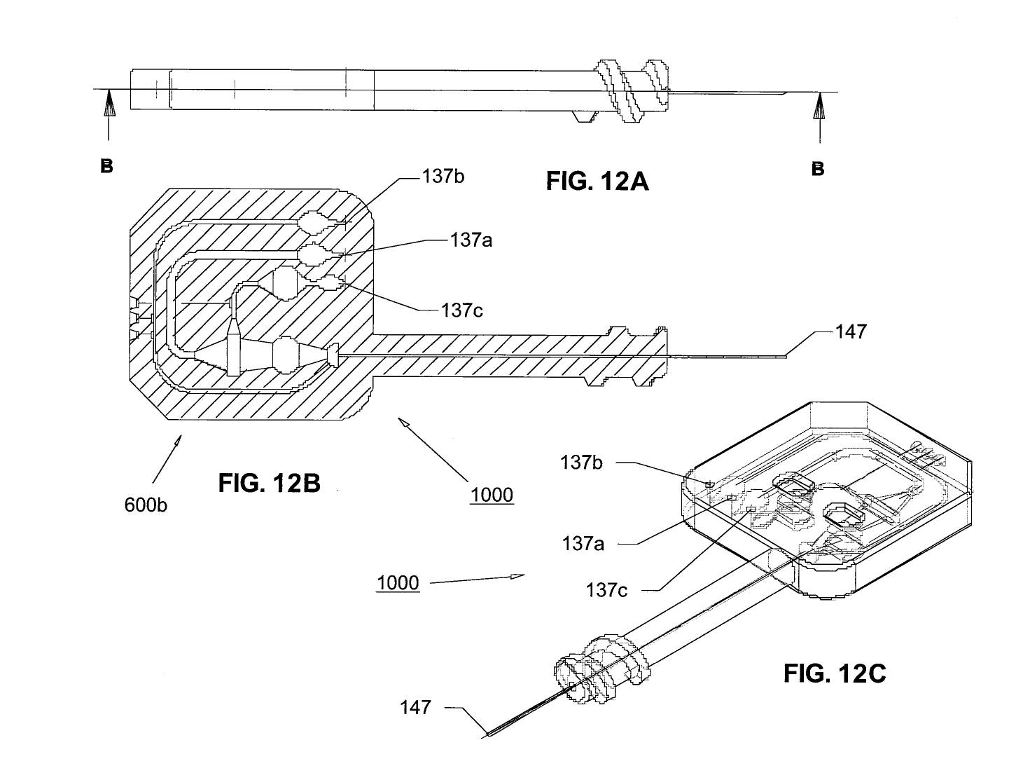

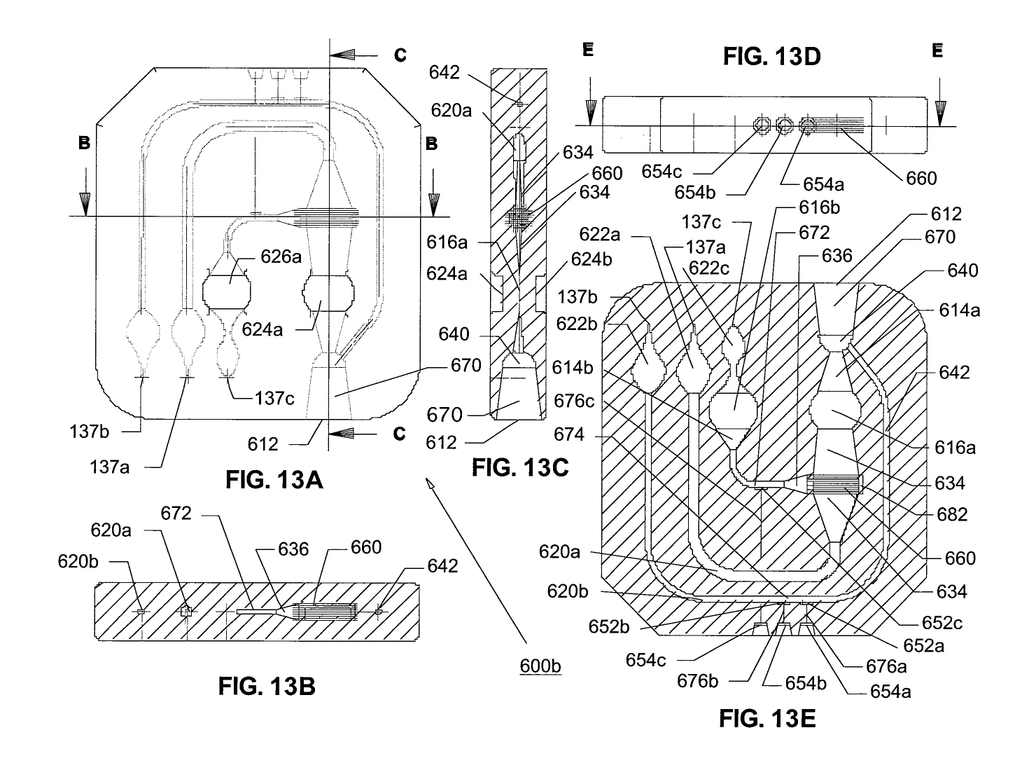

Referring to FIG. 12A, shown is a schematic drawing illustrating a top view of a second integrated needle and measurement apparatus 1000, for a hollow needle assembly according to a sixth embodiment of the invention; FIG. 12B illustrates a cross-sectional view through the apparatus shown in FIG. 12A along line B-B; FIG. 12C is a perspective view of the apparatus shown in FIG. 12A. Details of the measurement apparatus 600b are illustrated in FIG. 13A-E.

Referring to FIGS. 13A-E, shown are schematic drawings illustrating details of the measurement apparatus 600b shown in FIGS. 12A-C. The apparatus 600b is also a plasma extraction apparatus, and the measurement technology includes spectroscopy with the optional use of one or more than one reagent, and biosensors.

Referring to FIG. 13A is a top view of the apparatus 600b showing the sample inlet opening 612, the inlet chamber 670, a whole blood optical chamber wall-portion 624a, a plasma optical chamber wall-portion 626a, and three vents 137a, 137b, and 137c. The apparatus 600b contain two whole blood flow paths and one plasma flow path. The flow paths are illustrated in FIG. 13E.

Referring to FIG. 13B, shown is a cross-sectional view through apparatus 600b illustrated in FIG. 13A along line B-B, showing parts identified in FIG. 13E.

Referring to FIG. 13C, shown is a cross-sectional view through apparatus 600b illustrated in FIG. 13A along line C-C, showing parts identified in FIG. 13E.

Referring to FIG. 13D, shown is a rear view of apparatus 600b illustrated in FIG. 13A, showing the three electrical output contacts 654a, 654b, and 654c described in FIG. 13E.

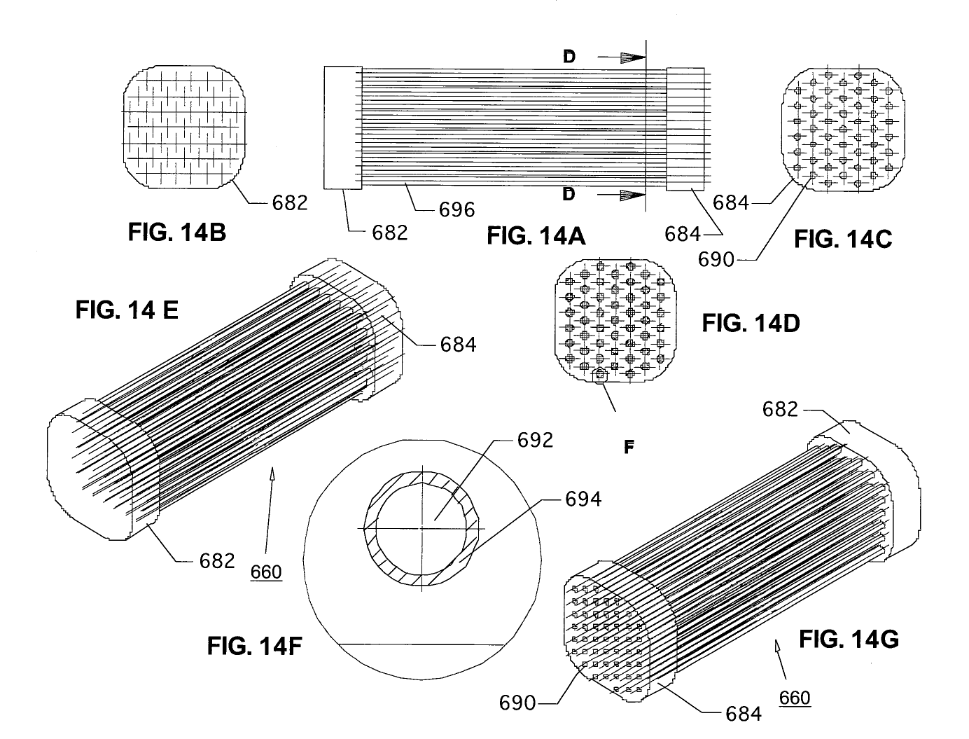

Referring to FIG. 13E, shown is a cross-sectional view through apparatus 600b illustrated in FIG. 13D along line E-E. Still referring to FIG. 13E, shown is the sample inlet opening 612, the inlet chamber 670. In use, as a non-limiting example, the blunt open end 171 of an apparatus 300 illustrated in FIGS. 5A, D, E, and F is first securely inserted into the inlet chamber 670 of the measurement apparatus 600b. Then the sharp open end 147 of the needle 100 is inserted into a blood vessel, allowing the blood to flow into the apparatus 600b, arriving at first at the manifold 640; from the manifold 640, the blood is distributed into the two whole blood flow paths: the blood biosensor flow path and the blood spectroscopy flow path. The blood biosensor flow path includes in series, the blood biosensor inlet transition chamber 642, the blood biosensor chamber 674, the blood biosensor outflow chamber 620b, the blood biosensor capillary break 622b, and terminating at the blood biosensor vent 137b. The blood spectroscopy flow path includes in series, the blood spectroscopic inlet transition chamber 614a, the blood optical chamber 616a, the filtration chamber 634 (for extracting plasma from the whole blood using the hollow fiber bundle 660 with closed flange 682; the hollow fiber bundle 660 is shown in details in FIGS. 14A-G), the filtration chamber outflow 620a, the filtration chamber capillary break 622a, and terminating at the filtration chamber vent 137a. A third flow path is defined as a plasma flow path, but is still in fluid connection with the sample inlet 612. The third flow path continues from the filtration chamber 634 at the plasma collection chamber 636, and includes in series the plasma biosensor chamber 672, the plasma spectroscopic inlet transition chamber 614b, the plasma optical chamber 616b, the plasma capillary break 622c, and terminating at the plasma vent 137c. One plasma biosensor is shown as 652c, which is electrically connected through a medium 676c to the electrical output contact 654c. Two whole blood biosensors are shown as 652a and 652b, which are connected to their respective electrical output contacts 654a and 654b, through respective media 676a and 676b. The pressure in the blood vessel is sufficient to force the blood into the measurement apparatus, via the needle, especially when the blood vessel is an artery. If the blood vessel is a vein, application of a tourniquet may be required in some patients.

Referring to FIGS. 14A-G, shown are schematic drawings illustrating details of the hollow fiber bundle 660 shown inside the plasma extraction chamber 634 illustrated in FIGS. 13B-E. The hollow fiber bundle 660 comprises several hollow fibers 696, held together by two flanges 682 and 684.

Referring to FIG. 14A, shown is a top view of the hollow fiber bundle 660, illustrating the closed flange 682, and the perforated flanged 684, and one hollow fiber 696; FIG. 14B illustrates a left side-view of the hollow fiber bundle 660, illustrating the closed flange 682; FIG. 14C illustrates a right side-view of the hollow fiber bundle 660, showing the perforated flange 684, and the open end 690 of a hollow fiber; FIG. 14D illustrates a cross-sectional view through the bundle 660 shown in FIG. 14A along line D-D; FIG. 14E illustrates a perspective view of the hollow fiber bundle 660, showing the closed flange 682; FIG. 14G illustrates an alternative perspective view of the hollow fiber bundle 660, showing the perforated flange 684, and the open end 690 of a hollow fiber 696. The hollow fibers are inserted inside perforations in the flange 684 and sealed at the juncture of the hollow fiber and the flange. Referring to FIG. 14F, shown is a detailed view of the cross-section of a hollow fiber, according to detail F identified in FIG. 14D, showing the lumen 692 of the fiber 696, and the wall of the fiber (also referred to as membrane) 694. In some embodiments, the walls of the hollow fiber (also referred to as hollow fiber filter) contain pores with an approximate distribution of diameters ranging from about 0.1 micrometer to about 10 micrometers, and the internal diameter of the hollow fiber ranges approximately from about 0.1 mm to about 1 mm. Those skilled in the art will appreciate that blood flow decreases the viscosity of the blood and therefore enhances separation (or filtration, or extraction) of plasma from blood; separation of plasma from blood also increases with increasing pore size, decreasing thickness of the membrane 694, and increasing membrane surface area. The surface area increases in proportion to the number of hollow fibers used in the bundle 660.

Referring to FIGS. 15A-C, shown are schematic drawings illustrating a measurement apparatus 600c suitable for attachment to a needle as illustrated in FIGS. 1A-F, via the internal threads in female receptor 163, and the matching threads in the inlet tubing 672 shown in FIGS. 15A-B; FIG. 15A illustrates a side view of the apparatus 600c; FIG. 15B illustrates a cross-sectional view through the apparatus 600c shown in FIG. 15A along line A-A; and FIG. 15C illustrates a perspective view of the apparatus 600c. The apparatus 600c illustrated in FIGS. 15A-C is similar to the apparatus 600a illustrated in FIGS. 13A-E, and accordingly, elements common to them share common reference numerals. The primary difference is that apparatus 600c does not have a filtration chamber for extracting plasma from whole blood.

Referring to FIGS. 16A-D, shown are schematic drawings illustrating a needle and barrel assembly 1100, with the sharp end 147 of the needle shaft 143 extended outside the barrel 200, for a hollow needle assembly according to a seventh embodiment of the invention; FIG. 16B illustrates a cross-sectional view through the apparatus shown in FIG. 16A along line B-B; FIG. 16C illustrates a perspective view of the apparatus shown in FIG. 16A; and FIG. 16D illustrates a detailed view of the detail D shown in FIG. 16B, illustrating the second embodiment of a flexible member 185. The apparatus 1100 illustrated in FIGS. 16A-D is similar to the apparatus 500 illustrated in FIGS. 7A-D, and accordingly, elements common to both share common reference numerals. The primary differences, illustrated in FIGS. 16A-D are: the absence of a spring; and the axis 133c of the back end of the hub running through the blunt open end 137, is different from axes 133a and 133b running through the sharp end 147. In this specific embodiment of the apparatus, the axis 133c is orthogonal to axes 133a and 133b.

Referring to FIG. 17A, shown is a schematic drawing illustrating a top view of the needle and barrel assembly 1200, with the sharp end 147 of the needle 100 extended outside the barrel 200, for a hollow needle assembly according to an eight embodiment of the invention; FIG. 17B illustrates a front view of the apparatus 1200 shown in FIG. 17A; and FIG. 17C illustrates a perspective view of the apparatus shown in FIG. 17A. The apparatus 1200 illustrated in FIGS. 17A-C is an assembly of a modified apparatus 400 illustrated in FIGS. 6A-D, and accordingly, elements common to these share common reference numerals. The primary difference, illustrated in FIGS. 17A-B, is a slot 113a with a hooked end 113b. The hooked end 113b is used for securing the stud 115, so that the needle cannot move relative to the barrel during insertion of the sharp end of the needle into a blood vessel, and pressing against the stud 115 is not necessary.

Referring to FIG. 18A, shown is a schematic drawing illustrating a top view of the needle and barrel assembly 1300, with the sharp end 147 of the needle 100 extended outside the barrel 200, for a hollow needle assembly according to a ninth embodiment of the invention; FIG. 18B illustrates a right side-view of the apparatus shown in FIG. 18A; and FIG. 18C illustrates a cross-sectional view through the apparatus shown in FIG. 18A along the line C-C. The apparatus 1300 illustrated in FIGS. 18A-C is an assembly of a modified needle 100 illustrated in FIGS. 1A-F, and a barrel 200 illustrated in FIGS. 2A-F and accordingly, elements common to these share common reference numerals. The primary difference, illustrated in FIGS. 8A and C, is that the female receptor 163 of the needle 100, can accept the male end 137b of a syringe 1500 illustrated in FIGS. 19A-C.

Referring to FIGS. 18c, the hollow needle assembly 1300 has a needle 100 with a shaft 143 with a length dimension I₁ and a hub 191 (with a front end 139 and a back end 123) with a length dimension I₄. In some aspects of the invention, the length dimension I₄ of the hub 191 is greater than the length dimension I₁ of the shaft 143 of the needle 100.

Referring to FIG. 19A, shown is a schematic drawing illustrating the apparatus 1300 (the ninth embodiment of the apparatus illustrated in FIGS. 18A-C) adjacent to a syringe 1500; FIG. 19B illustrates a cross-sectional view through the apparatus shown in FIG. 19A along the line B-B, showing the female receptor 163 in the hub of the needle, and the male end 137b of a syringe, that can mate with the female receptor 163; and FIG. 19C illustrates a perspective view of the syringe and needle, shown in FIG. 19A. The ninth embodiment of the invention can be used as an alternative needle for a traditional syringe, for minimizing the risks of needle-stick injuries.

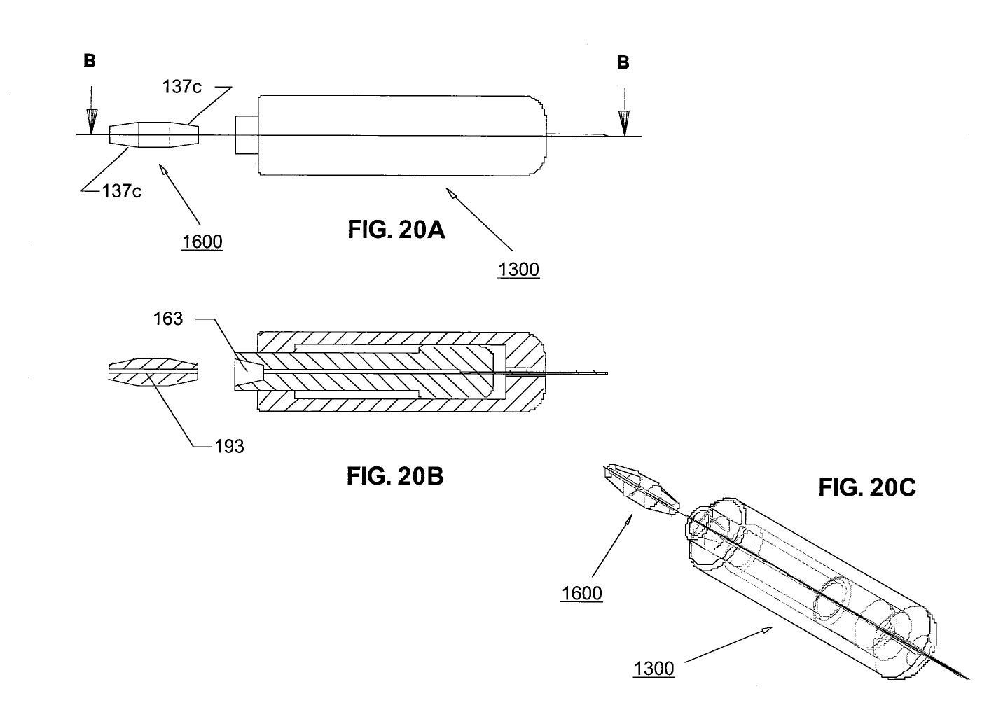

Referring to FIG. 20A, shown is a schematic drawing illustrating the apparatus 1300 (the ninth embodiment of the apparatus illustrated in FIGS. 18A-C) adjacent to an adaptor 1600 comprising two male ends 137c; FIG. 19B illustrates a cross-sectional view through the apparatus shown in FIG. 19A along the line B-B, showing the female receptor 163 in the hub of the needle, and the male ends 137c of the adaptor 1600 that can mate with the female receptor 163, producing an apparatus with a male blunt open end; and FIG. 19C illustrates a perspective view of the adaptor 1600 and needle, shown in FIG. 19A.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein.

1. A hollow needle assembly comprising:
a needle constructed of one or more than one part, the needle comprising a hub, wherein the hub comprises a back end with a blunt open end, a front end, and a passage, the needle further comprising a shaft having a sharp open end and a lumen, wherein the lumen is fluidly connected to the passage, and the needle further comprising a needle flow path defined along the lumen and the passage, beginning at the sharp open end and terminating at the blunt open end; and
a barrel constructed of one or more than one part, comprising an open anterior end through which a portion of the shaft of the needle passes, and an open posterior end through which a portion of the hub of the needle passes, the barrel further comprising an internal chamber for housing at least a portion of the needle.

2. A hollow needle assembly according to claim 1, wherein the barrel further comprises a slot through its wall, and the front end of the hub has a stud projecting into the slot of the barrel.

3. A hollow needle assembly according to claim 1, further comprising a safety cap, which fits over the open anterior end of the barrel when the sharp open end of the needle is retracted.

4. A hollow needle assembly according to claim 1, wherein the open anterior end is a first annular stop, and the open posterior end is a second annular stop.

5. A hollow needle assembly according to claim 1, wherein the front end of the hub is cylindrical having an outside diameter, and the internal chamber of the barrel is cylindrical having an inside diameter, and wherein the inside diameter is slightly larger than the outside diameter.

6. A hollow needle assembly according to claim 1, wherein the front end of the hub is cylindrical with external threads, and the internal chamber of the barrel is cylindrical with internal threads, wherein the external threads mate with the internal threads, the external threads and internal threads enabling extension and retraction of the shaft by rotating the barrel around the needle, and wherein movement of the front end of the hub is restricted to the portion of the barrel with threads.

7. A hollow needle assembly according to claim 1, wherein the back end of the hub protrudes through a locking cap, the locking cap is frictionally engaged to the open posterior end of the barrel, and the locking cap comprises a flexible member fitted inside the locking cap at the juncture of the inside of the locking cap and the open posterior end, permitting compression of the flexible member when the locking cap is pushed towards the sharp open end, thereby locking the needle in a position.

8. A hollow needle assembly according to claim 1, wherein the back end of the hub protrudes through a locking cap, the locking cap comprises internal threads and a flexible member fitted inside the locking cap at the juncture of the inside of the locking cap and the open posterior end, and the posterior end contains external threads, whereby the internal threads mate with the external threads, and compression of the flexible member is accomplished by screwing the posterior end into the locking cap, thereby locking the needle in position.

9. A hollow needle assembly according to claim 8, wherein a spring is fitted in the internal chamber of the barrel, around the shaft and between the front end of the hub and the open anterior end of the barrel.

10. A hollow needle assembly according to claim 1, wherein the back end of the hub houses a tapered projection which resembles the male end of a syringe, and wherein the tapered projection houses the blunt open end.

11. A hollow needle assembly according to claim 1, wherein the back end of the hub houses a female receptor, for receiving the male end of a syringe.

12. A hollow needle assembly according to claim 1, wherein the back end of the hub houses a female receptor, for receiving the male end of a measurement apparatus.

13. A hollow needle assembly according to claim 1, wherein the back end of the hub houses a male projection, for receiving the female end of a measurement apparatus.

14. A hollow needle assembly according to claim 1, wherein the back end of the hub houses a female receptor, for receiving one end of an adapter comprising two male ends, thereby transforming the female receptor into a male projection, resembling the male end of a syringe.

15. A hollow needle assembly according to claim 1, wherein the back end of the hub also includes a measurement apparatus comprising at least one flow path terminating at a vent, and wherein the at least one fluid path is in fluid connection with the passage and the lumen, and wherein the vent becomes the blunt open end.

16. A hollow needle assembly according to claim 1, wherein the fully extended shaft, outside the barrel, has a length that is in the approximate range of about 5 mm to about 30 mm.

17. A hollow needle assembly comprising:
a needle constructed of one or more than one part, the needle comprising a hub, wherein the hub comprises a back end with a blunt open end, a front end, and a passage, and the hub also having a first length dimension, the needle further comprising a shaft having a sharp open end and a lumen, and the shaft also having a second length dimension, wherein the lumen is fluidly connected to the passage, and the needle further comprising a needle flow path defined along the lumen and the passage, beginning at the sharp open end and terminating at the blunt open end, and wherein the first length dimension is greater than the second length dimension; and
a barrel constructed of one or more than one part, comprising an open anterior end through which a portion of the shaft of the needle passes, and an open posterior end through which a portion of the hub of the needle passes, the barrel further comprising an internal chamber for housing at least a portion of the needle.

18. A hollow needle assembly comprising:
a needle constructed of one or more than one part, the needle comprising a hub, wherein the hub comprises a back end with a blunt open end, a front end, and a passage, the needle further comprising a shaft having a sharp open end and a lumen, wherein the lumen is fluidly connected to the passage, and the shaft also having a first length dimension, and the needle further comprising a needle flow path defined along the lumen and the passage, beginning at the sharp open end and terminating at the blunt open end; and
a barrel constructed of one or more than one part, comprising an open anterior end through which a portion of the shaft of the needle passes, and an open posterior end through which a portion of the hub of the needle passes, the barrel further comprising an internal chamber for housing at least a portion of the needle, and the barrel also having a second length dimension, wherein the second length dimension is greater than the first length dimension.

19. A method of filling a syringe with blood comprising:
engaging the blunt open end of the hollow needle assembly according to claim 11, to the male end of a syringe;
extending the shaft of the needle of the hollow needle assembly;
piercing a vessel with the sharp open end of the needle of the hollow needle assembly;
allowing the blood to flow into the syringe, via the needle;
withdrawing the needle from the vessel; and
retracting the needle into the barrel.

20. A method of filling a measurement apparatus comprising at least one flow path beginning at an inlet opening and terminating at a vent, with blood from a vessel, comprising:
engaging the blunt open end of the hollow needle assembly according to claim 1, to the inlet opening of the measurement apparatus;
extending the shaft of the needle of the hollow needle assembly;
piercing the vessel with the sharp open end of the needle of the hollow needle assembly;
allowing the blood to flow into the measurement apparatus, via the needle;
withdrawing the needle from the vessel; and
retracting the needle into the barrel.
